(19) United States
(12) Patent Application Publication
(10) Pub. No.: US 2004/0162459 A1
(43) Pub. Date: Aug. 19, 2004

(54) Afterloader apparatus

(76) Inventors: Sam F. Liprie, Lake Charles, LA (US); Oleg Shikhman, Trumbull, CT (US)

Correspondence Address: CANTOR COLBURN LLP, 55 GRIFFIN ROAD SOUTH, BLOOMFIELD, CT, 06002, US

(21) Appl. No.: 10/773,730

(22) Filed: Feb. 5, 2004

Related U.S. Application Data
(63) Continuation of application No. 10/010,143, filed on Nov. 7, 2001.
Application No. 10/010,143 is a continuation-in-part of application No. 09/509,253, filed on Jun. 12, 2000, now Pat. No. 6,350,227.
Application No. 09/509,253 is a continuation of application No. PCT/US98/19820, filed on Sep. 23, 1998.
(60) Provisional application No. 60/059,602, filed on Sep. 23, 1997.
(60) Provisional application No. 60/246,873, filed on Nov. 8, 2000.

(51) Int. Cl. A61N 5/00

(52) U.S. Cl. 600/7

(57) ABSTRACT

A manually operable afterloader is provided for use during radiation therapy treatment of a patient. The afterloader includes at least one source wire drum, for the receipt of a non-radioactive portion of a source wire, and a shield capsule having a passageway therethrough for receipt of a radioactive portion of a source wire. A cranking mechanism is provided to rotate the source wire drum and drive the radioactive source wire out of the shield capsule.

### CROSS REFERENCE TO RELATED APPLICATIONS

The present application claims priority from U.S. patent application Ser. No. 10/010,143, filed Nov. 7, 2001, which claims priority to U.S. Provisional Patent Application Serial No. 60/246,873 and U.S. patent application Ser. No. 09/509,253, filed Mar. 23, 2000, which is a national stage filing of PCT/US98/19820, filed Sep. 23, 1998, which relies on U.S. Provisional Patent Application Serial No. 60/059,602, filed Sep. 23, 1997 for priority, each of the disclosures of which are specifically incorporated herein by reference in their entireties.

### BACKGROUND

Radiation is used to treat various diseases of the body, including among others, cancer in various areas of the body and sites of stenosis within the vasculature (whether following angioplasty or otherwise). Such therapy will generally be described herein as “brachytherapy.” “Angioplasty” will be used generally to describe the opening of a constriction in a blood vessel or artery. The following disclosure will discuss applications to vascular brachytherapy, though it should be realized that the present disclosure is equally applicable to other types of brachytherapy, including treatment of diseases, with particular emphasis on stenoses, within other bodily conduits.

Radiation treatment has found particular application in vascular brachytherapy for the prevention of re-closure or restenosis of vascular constriction. It has been proposed that where transcatheter procedures tear vascular endothelial lining, smooth muscle cells (SMC's) are stimulated to divide. Phil et al., “The Basic Radiobiology of Intravascular Irradiation,” From Waksman R. (ed.). Vascular Brachytherapy, Second Edition. Armonk, N.Y.: Futura Publishing Co., Inc.; © 1999, pp. 65-66. The stimulated SMC's pour through the tear in the endothelial lining caused by the transcatheter procedure forming neointima, thereby restenosing the vessel. Id. A significant reduction in size of neointima, typically caused by unchecked smooth muscle proliferation, has been observed incident to balloon angioplasty where the procedure is immediately preceded or followed by intracoronary irradiation of the targeted tissues. Wilcox et al., “Mechanisms by Which Radiation May Prevent Restenosis: Inhibition of Cell Proliferation and Vascular Remodeling,” From Waksman R. (ed.). Vascular Brachytherapy, Second Edition. Armonk, N.Y.: Futura Publishing Co., Inc.; © 1999, p. 127.

During vascular brachytherapy, a radioactive source (or sources) is positioned, usually through a catheter, at the site of the lesion to be treated. The source then irradiates the surrounding tissue from one or more positions for specific durations of time calculated to be optimally effective for prevention of restenosis of the lesion. The source is withdrawn from the patient into a protective container for safe handling.

Afterloaders are devices generally used to accurately advance and retract a flexible drive member containing the radioactive source over a specified distance for a specific period of time. An afterloader generally consists of a flexible simulation (test) drive member, a flexible drive member containing a radioactive element or source wire, computer controllers and motorized drive mechanisms to operate both types of flexible members, a shielding safe for the radioactive element, an internal timer, and in respiratory brachytherapy, an exit port attached to a rotating wheel that allows multiple transport tubes (previously placed into the patient) to be hooked up to the device at the same time.

The afterloader may send out the simulation member to check the patency of the transport tube without subjecting the patient to undue radiation exposure, and then may send out the radioactive element. After treatment is performed in the first transport tube, the afterloader retracts the source into the shielding safe inside the afterloader, and a wheel turns to align a slot containing the second transport tube to an exit port. The afterloader then repeats its function, sending and retracting the simulation member and radioactive member through this second tube. The procedure is repeated until the function is carried out through all the specified transport tubes. Since afterloaders use a fixed, short length radioactive source, the afterloaders must multi-step this source many times inside each transport tube to cover the diseased area.

Current remote afterloaders, initially designed for respiratory brachytherapy, are particularly complicated. Limiting factors of prior art afterloaders are the physical size and amount of equipment necessary to operate a remote afterloader. In many treatment facilities, there is not enough room for the required size and amount of equipment. Thus there exists a need for a simple, compact, portable, self-contained afterloader for use in conjunction with, or after, an angioplasty procedure to provide radiation treatment of a vessel for the prevention of restenosis.

### SUMMARY

The above discussed and other drawbacks and deficiencies of the prior art are overcome or alleviated by the present afterloader, including a flexible drive member housing a radioactive source in a distal end. In a parked position, the distal end of the flexible drive member is contained within a radioactive shielding member.

The flexible drive member is connected at a proximal end to a drive shaft or wheel (e.g., drum) operable by a manual cranking mechanism. A guide tube may be provided between the source wire drum and the pathway of the containment capsule. An adjustable structure, or an adjustment clamp, may also be provided about the guide tube to align one end of the guide tube with a tangent of the source wire drum so as to receive the source wire from the drum.

The afterloader may include a simulation wire connected to a second drive shaft or wheel operable by a second manual cranking mechanism or operable by selective engagement with the first manual cranking mechanism. A guide tube may be provided between the simulation wire drum and a wire union bracket and feed plate. An adjustable structure, or an adjustment clamp, may also be provided about the guide tube to align one end of the guide tube with a tangent of the simulation wire drum so as to receive the simulation wire from the drum.

The manual cranking mechanism or mechanisms are operably engageable with the source wire and simulation wire drums. A cranking mechanism generally includes a crank wheel having a crank handle attached thereto. The crank wheel is connected to a drive shaft, which in turn may be connected to one side of a slip clutch. A wire drum is mounted on a second shaft, which may be connected to the opposed side of the slip clutch. Thus, rotation of the crank wheel by a manipulation of the crank handle rotates the respective drive shafts through the clutch to drive the wire on and off of the wire drum.

Further, as a safety mechanism, the crank handle may be pivotally mounted to the crank wheel such that when the crank handle is in a retracted position, it engages a frictional safety, thereby preventing inadvertent rotation of the crank wheel and wire drum. A separate braking mechanism may also be provided on or adjacent the crank to additionally limit undesired rotation of the wire drum.

The wire drum may take any configuration that will spool and unspool a wire. The drum itself may be smooth, or optionally, it may include one or more circumferential grooves for receipt of the wire. Further, a flexible cable or belt may be provided over the drum to restrain the wire against the drum as the drum is rotated, so that the wire does not flex or pop off the drum as should the wire encounter resistive forces going into a treatment catheter.

Clean spooling and unspooling of the wire may also be assisted by pairs of tension wheels, which control the movement of the flexible cable and provide predetermined rates of tension on the cable so as to restrain the wire against the drum (or within a groove on the drum). In an exemplary embodiment, the takeup/tension assembly may include a first pulley fixedly mounted to a vertical main plate and adjacent the wire and a floating pulley, floatingly mounted and biased by spring tension. An adjustment mechanism may be provided to adjust the tension that the flexible pulley provides on the cable associated with the flexible pulley.

### BRIEF DESCRIPTION OF THE DRAWINGS

Various embodiments of the presently disclosed afterloader apparatus are described herein with reference to the drawing figures wherein:

FIG. 1 is an expanded overhead view of an exemplary embodiment of a multiple wire afterloader;

FIG. 2 is a cross-sectional side view of an exemplary drum, cable and tension wheel arrangement of an afterloader embodiment;

FIG. 2A is a cross-sectional side view of an exemplary fixed tension wheel and wire drum arrangement;

FIG. 3 is an overhead view of a temporary braking mechanism as applied to one drum of an afterloader mechanism;

FIG. 4 is an overhead view of an exemplary embodiment of a multiple wire afterloader;

FIG. 5 is a side view of an exemplary wire drum of an afterloader apparatus;

FIG. 6 is a top view of the wire drum of FIG. 5;

FIG. 7 is a side view of an exemplary wire drum illustrating the side opposite to that illustrated in FIG. 5;

FIG. 8 is a cross-sectional view taken along section line 6-6 of FIG. 7;

FIG. 9 is a cross-sectional view taken along section line 7-7 of FIG. 7;

FIG. 10 is an enlarged top view of an exemplary source wire drum;

FIG. 11 is a cross-sectional view of the source wire drum of FIG. 10;

FIG. 12 is an enlarged view of the indicated area of detail shown in FIG. 11, which shows an exemplary threaded groove configuration;

FIG. 13 is an exemplary alternative embodiment of the threaded groove configuration of FIG. 12;

FIG. 14 is a partial cross-sectional view of an exemplary alternative embodiment for retaining the source wire on the source wire drum;

FIG. 15 is a partial cross-section view illustrating an exemplary payout passageway of the source wire from the source wire drum according to the exemplary alternative embodiment of FIG. 14;

FIG. 16 is an overhead view of an exemplary afterloader casing and user interface;

FIG. 17 is an overhead view of an exemplary manual afterloader;

FIG. 18 is a side view of an exemplary lock mechanism and wire drum arrangement;

FIG. 19 is a side view of another exemplary lock mechanism and wire drum arrangement; and

FIG. 20 is a side view of another exemplary lock mechanism and wire drum arrangement.

### DETAILED DESCRIPTION OF THE PREFERRED EMBODIMENT

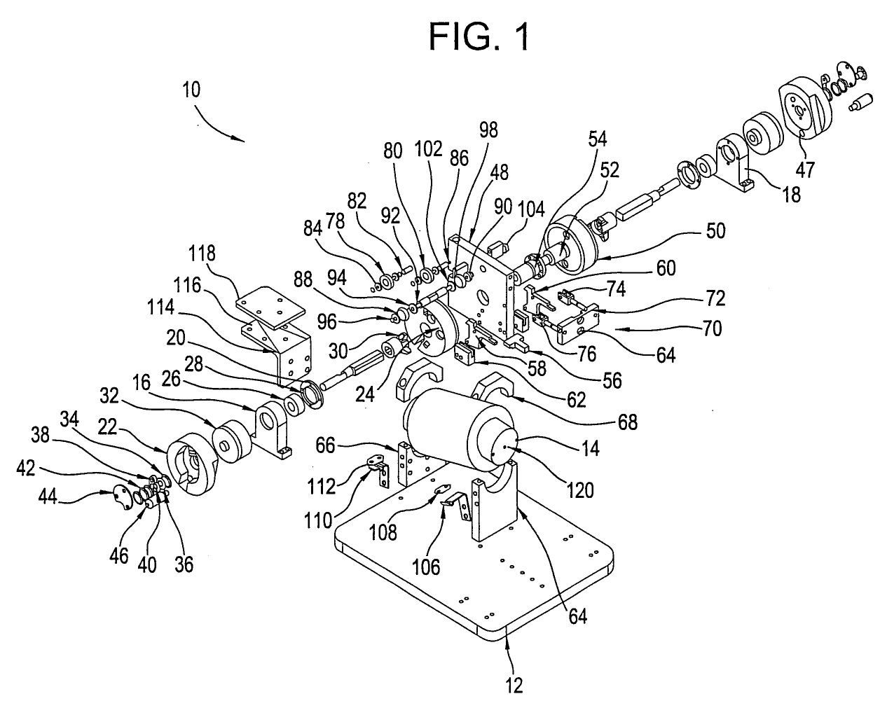

Referring now in detail to the respective FIGURES, in which like reference numerals identify similar or identical elements, a first embodiment of an exemplary afterloader apparatus is illustrated generally at 10 in FIG. 1. Briefly, a general function of the afterloader 10 is to deliver a source wire having a radioactive source contained near a distal end to a treatment site within a vessel of a patient (e.g., to prevent the restenosis of the vessel after an angioplasty procedure). The afterloader 10 drives the source wire (not shown) through a treatment catheter (not shown), which is attached to the afterloader 10 and extends to the treatment site within the vessel. The frame components of the afterloader 10 may be made from any materials, and in an exemplary embodiment are made of tool grade aluminum or steel. Exemplary materials used for the various other components of the afterloader 10 will be identified as appropriate.

Referring initially to FIG. 1, a base plate 12 mounts a containment capsule 14 which is configured to shield the radioactive portion of a flexible member (not shown). Thus, the containment capsule 14 comprises a material having physical characteristics and a geometry that is effective to prevent or attenuate the escape of radiation from a radioactive source.

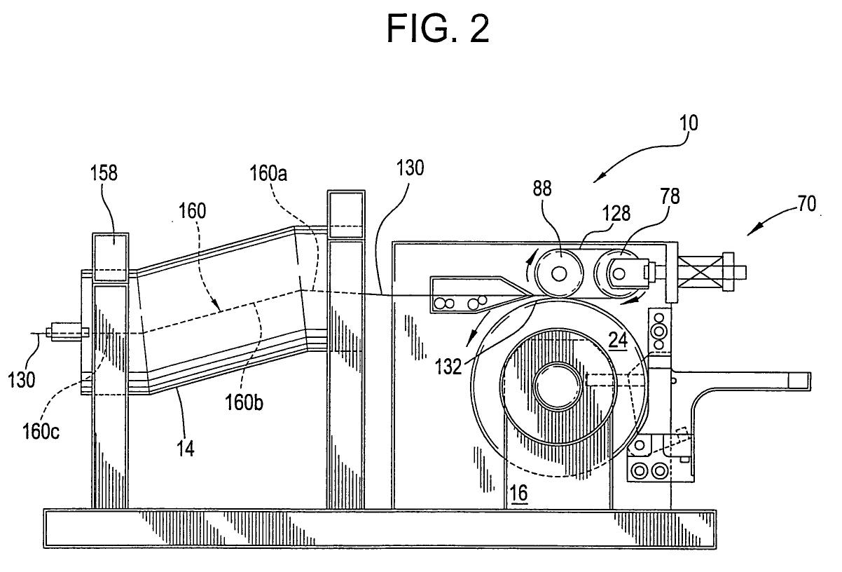

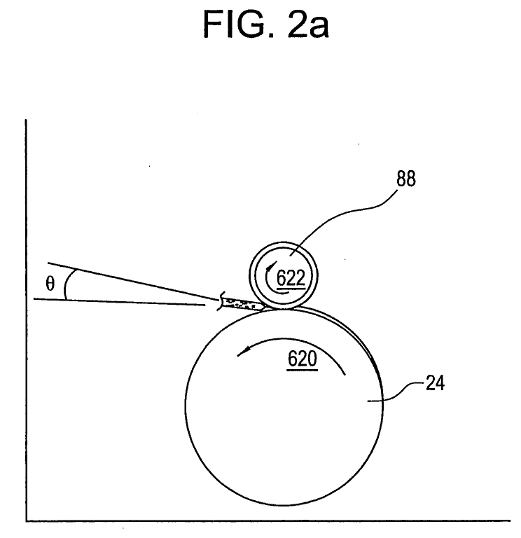

In one embodiment, the containment capsule prevents the escape of gamma radiation, which can only travel in a linear or line of sight direction, from a radioactive source contained at the distal end of a source wire. In an exemplary embodiment, the containment capsule 14 defines a pathway that is non-linear, as is shown generally at 160 in FIG. 2. Exemplary source wire pathways will be discussed in further detail below with reference to FIGS. 1 and 2.

Referring again to the exemplary embodiment of FIG. 1, first and second bearing housings 16, 18 are mounted to the base plate 12. A first drive shaft 20 generally connects a first crank 22 to a first wire drum 24. The first drive shaft 20 interfaces with the first bearing housing 16 via a shaft bearing 26 and a bearing retainer 28. The first drive shaft 20 interfaces with the first wire drum 24 via a first shaft interface bracket 30. The first drive shaft 20 extends through the first bearing housing 16 and through a first slip clutch 32 mated with a crank 22. The slip clutch 32 may be any suitable type, including without limitation, a Berg model no. JCO-4 (one direction) slip clutch. In an exemplary embodiment, the clutch 32 is rated to slip at approximately two pounds of force to limit the drive and retraction forces provided to the wire. Such an exemplary slip rating may be desirable to prevent the wire from advancing through the walls of the treatment catheter and to prevent retraction of the wire if stuck within the treatment catheter. While an approximate slip rating has been exemplified, other slip ratings may be more or less suitable depending on the forces encountered by the advancing or retracting wire for any given path and depending on the durability of the catheter and the pushability of the wire.

In another exemplary embodiment, a two-way clutch is utilized to provide the user with a positive indication at the extreme limits of travel for the wire. The clutch mechanism may also includes a ratchet type device, which provides tactile and/or audible indication of slippage. Also the clutch may be set to require the wire to move at an initially decreased rate (e.g., ramping) to prevent initial excessive forces on the wire. Referring again to the exemplary embodiment of FIG. 1, the crank 22 is held to the first drive shaft 20 by a retaining ring 34, followed by a ratchet wheel 36, a deep cable clamp 38, a ratchet holder 40, and a support washer 42. A ratchet cover 44 is provided over the support washer 42 within the crank 22. Provided on the crank 22, off-center from the longitudinal axis of first drive shaft 20, is a rotating handle 46 for turning the crank 22. The handle 46 is configured to fold inwardly towards the crank 22, thereby engaging a temporary brake (the brake shown in a retracted position as 47 in relation to a preferred second housing wall 18 in FIG. 1).

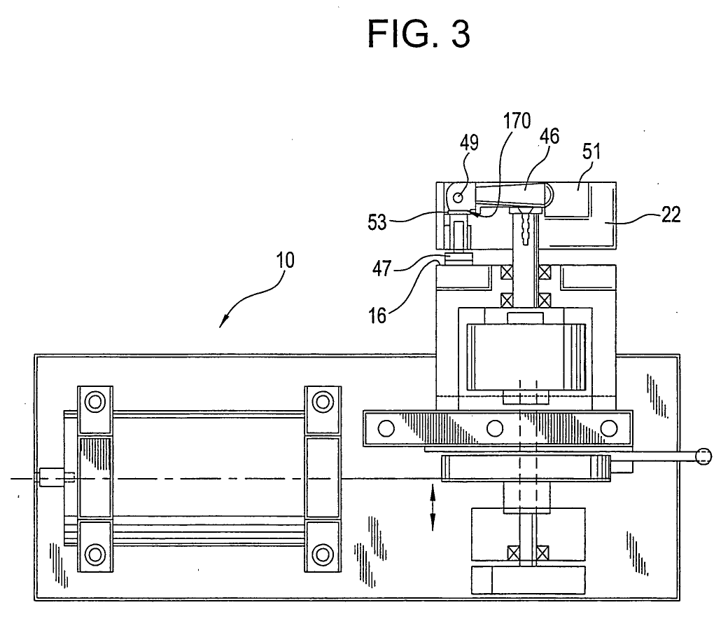

In the illustrated exemplary embodiment as shown in FIG. 3, the crank handle 46 is pivotally attached to the crank 22 by a pivot pin 49 and is movable between a retracted position, wherein the crank handle 46 is disposed in a recess 51 formed in the crank 22, and an operative position, as shown in FIG. 1, wherein the crank handle 46 is rotated approximately 90 degrees outwardly from its retracted position. A curved camming surface 53 is provided on the crank handle 46 to interact with a stop member 47, which is preferably spring loaded to be biased in a retracted orientation such that when the crank handle 46 is in the operative position, the stop member is biased away from the housing wall 16. The stop member 47 is biased against the housing wall 16 by a side surface 170 of the crank handle when the crank handle is in the retracted position as illustrated in FIG. 3. In this manner, the stop member 47 frictionally engages the housing wall 16 to prevent inadvertent operation of the crank 22. In an exemplary embodiment, the stop member 47 comprises a material having a high coefficient of friction.

Referring again to FIG. 1, a vertical main plate 48 is mounted in a central position on the base plate 12 and provides support for the first and second wire drums 24, 50 and the surrounding afterloader components. Connectivity of the wire drums 24, 50 to the vertical main plate 48 is best seen with reference to the second wire drum 50. The second wire drum 50 is mounted on a drum-mounting shaft 52, which interfaces with the vertical main plate 48 at 54.

In the exemplary embodiment of FIG. 1, a releasable brake mechanism, or stop release lever, includes a swing stop handle 56 and swing stops 58, 60, is pivotally mounted to stop pivot blocks 62, 64. Stop pivot blocks 62, 64 are securely mounted to the vertical main plate 48. In an exemplary embodiment, the swing stops 56, 58 are spring biased toward an engaged orientation with first and second wire drums 24, 50, respectively. The swing stops 58, 60 limit rotation of the wire drums 24, 50 to a predetermined initial amount so that a source wire (not shown) can initially only advance a predetermined distance. While the pre-determined distance (or distances) may be set anywhere along the length of the wire (for example, to allow stepping of a source along a treatment zone), the predetermined distance, in one exemplary embodiment, is chosen to allow a distal end of the source wire to advance to within 3-5 centimeters of the distal end of a blind lumen in an associated treatment catheter. This prevents the source wire from hitting or piercing the distal end of the blind lumen and allows, when the braking mechanism is released, fine tuning of the position of the radioactive source on the distal end of the source wire within the treatment zone of the catheter. Such fine tuning may be accomplished, for example, with the aid of fluoroscopy. The swing stop lever 56 may be pivoted to a disengaged position, thereby disconnecting the swing stops 58, 60 from the wire drums 24, 50 to allow a further limited amount of source wire advancement. Referring still to the exemplary embodiment in FIG. 1, a tension yoke assembly 70 is mounted to a portion of the vertical main plate 48 and includes a tension mounting plate 72 and first and second tension wheel supports 74, 76. A first floating set of tension wheels includes first and second cable tension wheels 78, 80, which engage the first and second tension wheel supports 74, 76 and are biased against the first and second wire drums 24, 50, respectively. Each cable tension wheel 78, 80 is shown to be connected to a tension wheel support 74, 76 by a shaft 82, a yoke bearing 84 and a retaining ring 86. The first floating set of tension wheel supports 74, 76 are further spring biased against the tension mounting plate 72 to provide variable tension on the flexible cable.

Referring still to the exemplary embodiment of FIG. 1, a second fixed set of tension wheels, including third and fourth tension wheels 88, 90 is positioned on the vertical main plate 48 above the first and second wire drums 24, 50, respectively such that the second set of tension wheels is biased against the drums at or near the point at which the wires leave the drums. Each of the third and fourth tension wheels 88, 90 are connected to a main shaft 92 by a cable tension wheel bearing 94 and a cable tension wheel washer 96. The main shaft 92 is disposed through the vertical main plate 48. First and second wire retaining means, which non-limiting exemplary embodiments include flexible belts or cables, among others, may be provided to securely retain the flexible wires on the wire drums 24, 50 (A first wire retaining cable 128 is shown positioned around first and second cable tension wheels 78, 80 and biased to a first wire drum 24 in FIG. 2). In order to payout a wire from wire drums 24 or 50, a tangent opening (shown as 132 in FIG. 2) is provided by the tension yoke assembly 70 and first and second cable tension wheels 78, 80, which serves to divert the cable 128 from the wire drum 24, 50 temporarily to allow a gap to be formed at the tangent opening, thus permitting the wire to exit the wire drum 24, 50 tangentially and enter a guide tube, which as noted above, may be adjusted to have an opening aligned with a tangent coming off the tangent opening 132.

Referring now to FIG. 2a, an exemplary fixed tension wheel 88 is illustrated in relation to an exemplary wire drum 24. Wire payout rotation of the wire drum is illustrated by the arrow 620 while payout rotation of the fixed tension wheel is illustrated by the arrow 622. Exemplary wire 130 is shown paying out between the fixed tension wheel 88 and wire drum 24. In another exemplary embodiment, the wire pays out at an angle θ different from that of the tangent defined by the path between the fixed tension wheel 88 and the wire drum 24.

Referring again to FIG. 2, a first end of the first cable 128 is secured to the wire drum 24 with a clamp (described below). Cable 128 is wound over the source wire 130, and is deflected away from the source wire 130 onto the first and second cable tension wheels 78, 80 and back to a second clamp on the wire drum 24.

Referring again to the exemplary embodiment of FIG. 1, forward of the second, fixed set of tension wheels 88, 90, also disposed on the vertical main plate 48, are first and second tube clamp blocks 98, 100, including first and second tube clamps 102, 104. The tube clamps 102, 104 receive tubes (not shown), which serve as guides for the wires as they leave the first and second wire drums 24, 50, respectively. The guide tube clamps 102, 104 are aligned with a tangent of the wire drums 24, 50 and define paths along which the wires pay out from the wire drums 24, 50. The guide tube clamps 102, 104 are adjustable to align the end of the guide tubes with the wires retained on the wire drums 24, 50. The components of the guide tube clamps 102, 104 are thereafter tightened to fix the alignment of the guide tubes relative to the wire paths.

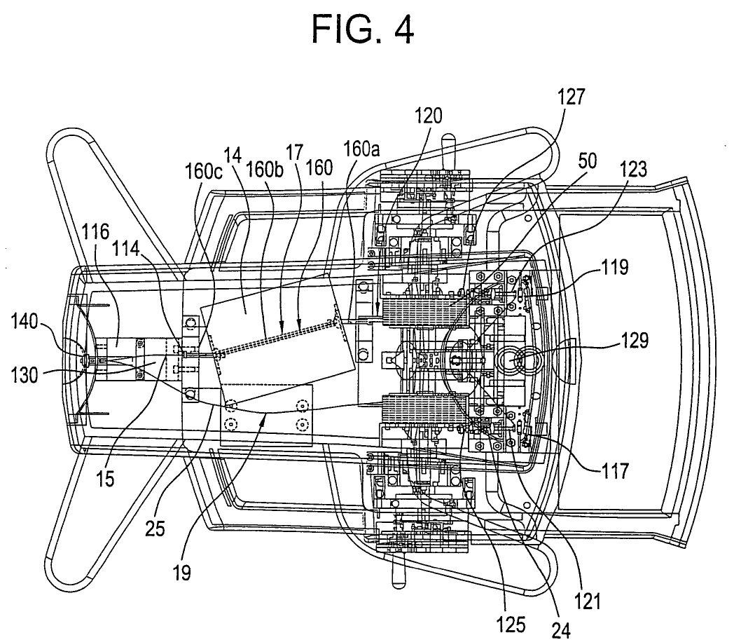

Referring now to FIGS. 1 and 4, two distinct wire paths are described by an exemplary afterloader embodiment. The illustrated first wire drum 24 is set to deploy a test, or dummy, wire. The test wire 25 plays off the first wire drum 24 between the third tension wheel 88 and the first tube clamp 102 (see FIG. 1). The test wire enters a first tube (not shown) held by the first tube clamp 102. The first tube traces a first wire path 19 between the first tube clamp 102, a third tube clamp 106 with cable clamp 108 and a fourth tube clamp 110 with cable clamp 112. At the fourth tube clamp 110, the wire feeds into a recess, shown generally at 114, between a wire union bracket 116 and a wire feed plate 118. The wire union bracket 116 mounts to the short capsule block 66 such that the recess 114 is positioned between the fourth tube clamp 110 and the containment capsule 14.

The second path 17 traces between the second (source wire) drum 50 and the recess 114 of the wire union bracket 116. The source wire 130 plays off the second wire drum 50 between the fourth tension wheel 90 and the second tube clamp 104. The test wire enters a second tube (not shown) held by the second tube clamp 104. The second tube traces a source wire path between the second tube clamp 104 and a first opening, shown generally at 120, in the containment capsule 14.

Referring now to FIGS. 2 and 4, as described above, an exemplary embodiment of the containment capsule 14 describes a nonlinear source wire path 15 terminating at the recess 114 of the wire union bracket 116. In particular the pathway 15 includes linear segments 160a, 160b and 160c, which are in communication such that they form a continuous non-linear pathway 160 through the containment capsule 14. When the radioactive source is positioned at the mid-point of the containment capsule 14, radiation is prevented from escaping out the open ends of the pathway 160 due to the non-linear configuration of the pathway 160. Thus, the emission of radiation is prevented because that radiation is limited to line of sight movement. While interconnected linear segments are disclosed, to form the pathway 160, it is contemplated that other non-linear configurations, such as for example, curvilinear, may be used as long as the pathway allows unimpeded travel of the source wire therethrough.

At the outlet of pathway 160c, the source wire feeds into a recess, shown generally at 114, between a wire union bracket 116 and a wire feed plate 118. At the outlet of the wire feed plate 118, a connector member 140, such as a Luer connector, is provided in order to connect the wire feed plate 118 with a treatment catheter (not shown). In an exemplary embodiment, the connector 140 is a proprietary type connector configured to mate with a correspondingly configured connector on a treatment catheter or extension tube.

Referring still to FIG. 4, exemplary first and second drum locks 117, 119 are evident. In an exemplary embodiment, the first and second drum locks are driven by a selector switch 129 (in an exemplary embodiment, an electrical key switch) (described below with regard to FIG. 16). In one embodimenthe first and second drum locks 117, 119 include first and second mechanical pistons 121, 123, which interface respectively with locking ports 125, 127 on the first and second drums 24, 50. The drum locks ensure that only one drum is actuated at a time according to the exemplary illustrated selector switch 129.

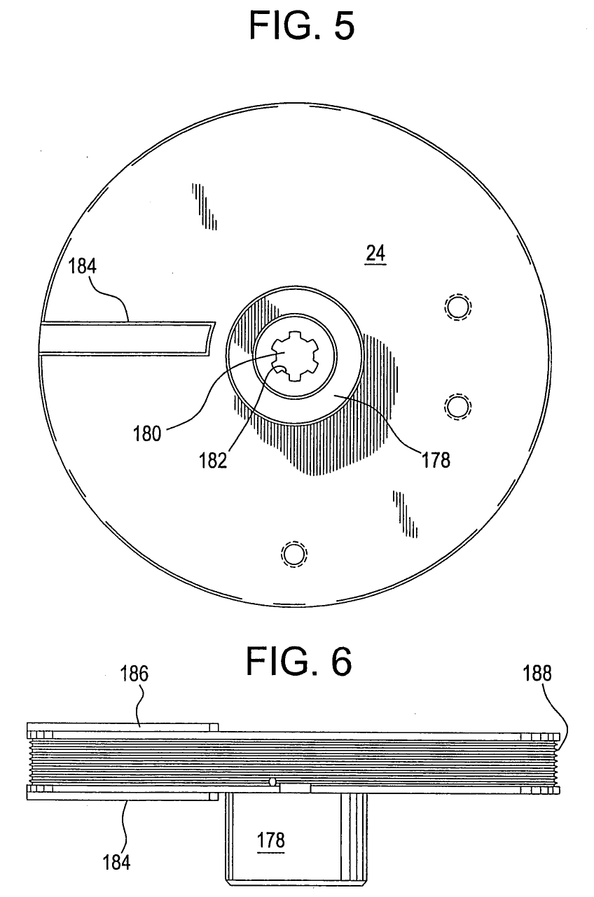

Referring now to FIGS. 5-6, specific details of an exemplary embodiment of the wire drums 24, 50 will now be addressed in greater detail. A wire drum 24, 50 includes a cylindrical hub 178, which surrounds a mounting bore 180. The mounting bore 180 is configured and dimensioned to securely receive the drum-mounting shaft 52 (see FIG. 1). In one embodiment, the mounting bore 180 is provided with gear teeth 182, which mesh with complementary gear teeth formed on the drum mounting shaft 52 to ensure that no slippage occurs between the wire drum 24, 50 and the shaft 52. A pair of stop members 184, 186 is provided on the outer surfaces of the wire drum. The stop members 184, 186 prevent the wire drum 24, 50 from being rotated so far that the wire detaches from the drum 24, 50. Stop member 184 limits the amount of rotation of the wire drum in a first direction, and thus the distance the wire travels in a first direction. Stop member 184 similarly limits the amount of rotation of the wire drum 24, 50 in a second direction, and thus limits the distance the wire travels in a second direction. In an exemplary embodiment, the stop members are of a material that is resilient and shock absorbent, such as rubber, an mix of a metal plate and rubber, a metal or other hydraulic piece, or a stiff spring, among others.

With reference to FIG. 6, another exemplary wire drum includes a threaded groove 188, which is formed around the outer perimeter of the wire drum 24, 50. The threaded groove 188 receives both the wire 130 and the flexible belt 128.

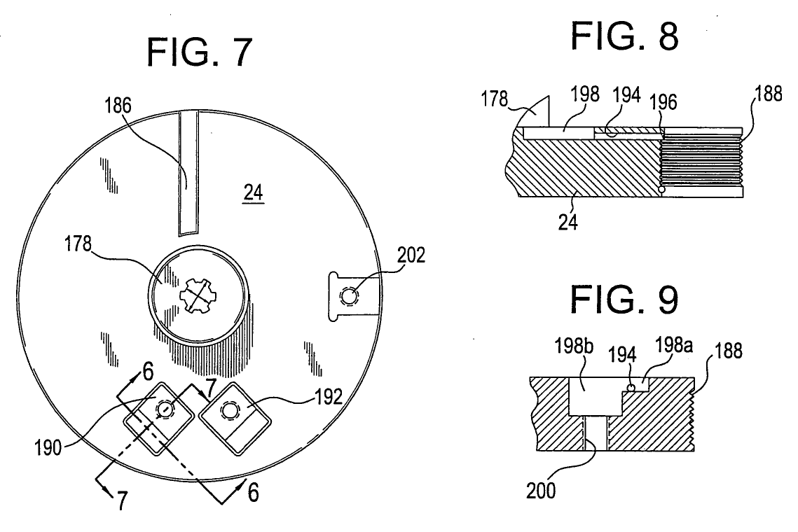

Referring now to FIG. 7, in order to retain the first or most proximal ends of the wire 130 and the flexible belt 128, a pair of clamps 190, 192 may be provided on the wire drum 24, 50. The operation of the clamps 190, 192 will be explained with reference to FIGS. 8-9, which show the details of clamp 190. The clamp 192 operates in the same manner.

An exemplary embodiment is shown in FIG. 8, wherein the wire drum 24 is provided with a bore 194 formed to extend from a terminus 196 of the exemplary threaded groove 188 to a recess 198 formed in the surface of the wire drum 24 to a radial distance inwardly from the outer edge of the wire drum 24. In this manner, the wire 130 or flexible belt 128, as appropriate, is fed through the bore 194 into the recess 198. Thereafter, clamp 190 is threadably secured into the recess 198 to secure a respective end of the wire 130 or belt 128 within the recess 198.

As shown in FIG. 9, in an exemplary embodiment, the recess 198 is formed as a stepped recess, having an upper shelf portion 198a and a lower shelf portion 198b. A threaded bore 200 is provided in communication with the lower shelf portion 198b. Clamp 190 is shown as being configured and dimensioned to the same configuration of the recess 198 with slightly smaller dimensions, such that it will fit therein. Clamp 190 is secured in the recess 198 by a threaded screw (not shown).

Referring to FIG. 7, a transport clamp 202 may be provided to retain the outer end of the flexible belt 128 during transport of the wire drum 24. This may facilitate replacement of a source wire 130 on the afterloader 10 with a new source wire when the radioactive portion of the source wire has decayed to a predetermined level of radioactivity.

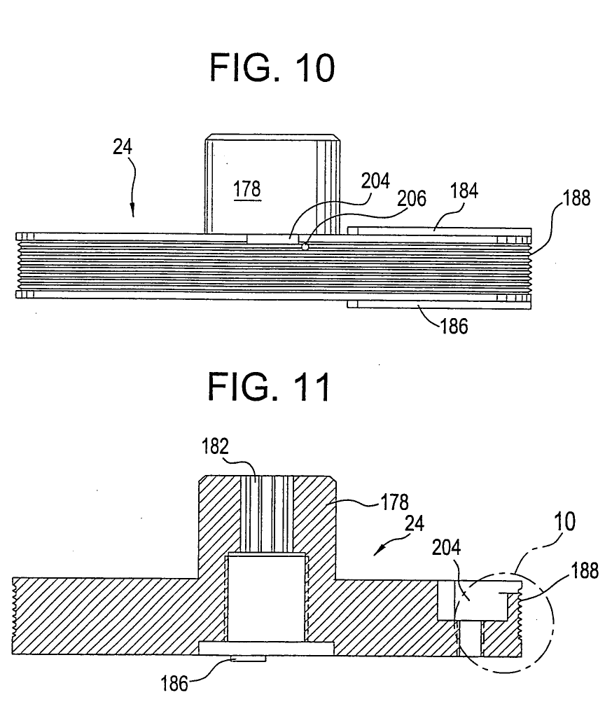

Referring to FIGS. 10-11, the illustrated transport clamp 202 fits within a recess 204 formed in the wire drum 24. The clamp 202 functions in a similar manner as clamps 190 and 192. For example, a bore 206 (FIG. 10) is formed radially inwardly through the wire drum 24 at the beginning of the exemplary threaded groove 188. The bore 206 is in communication with the recess 204, so that the transport clamp 202 will secure the end of the flexible belt 128 therein.

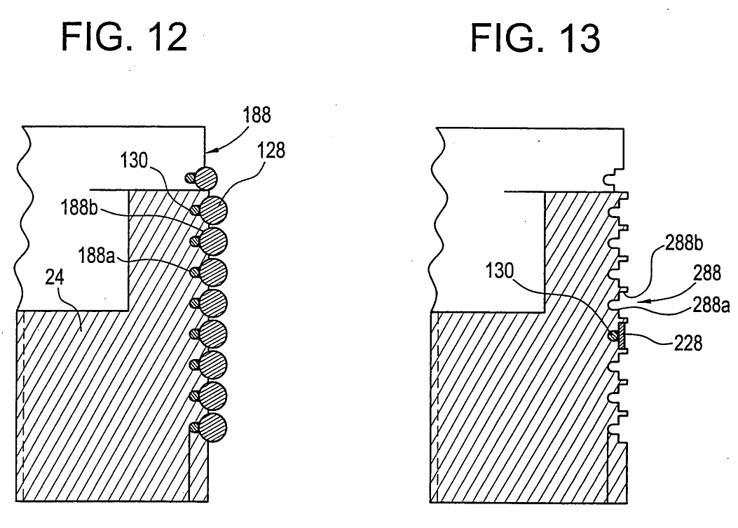

Referring now to FIG. 12, an exemplary wrapping arrangement of the flexible belt 128 around the wire drum 24 to retain the wire within the exemplary threaded groove 188 will now be described in detail. The threaded groove 188 is illustrated as a compound groove 188 having a lower groove portion 188a formed directly radially inwardly of an outer groove portion 188b. The wire 130 is wrapped onto the wire drum 24 within the lower groove portion 188a, and the cable 128 is wrapped around the wire drum 24 over the wire 130. The cable 128 is retained in the outer groove portion 188b. As illustrated in FIG. 12, in another exemplary embodiment, the diameter of the cable 128 is significantly larger than that of the wire, such that, upon paying out of the wire from the wire drum 24, the cable 128 prevents resistive forces, which are created as the wire navigates the numerous tortous pathways of the catheter positioned at a treatment site. These resistive forces may otherwise tend to force the wire 130 out of the threaded groove 188 or otherwise off the surface of the wire drum 24.

Referring now to FIG. 13, another exemplary embodiment illustrates a wire-retaining threaded groove 288, which includes an arcuate lower portion 288 and a squared-off upper portion 288b. The wire 130 is retained in the lower portion 288a by a cable 228 having a rectangular cross-section. It is within the scope of the present disclosure that numerous and different geometries may be utilized for the cables 128 for both grooved and groove less (e.g. a cable including a groove laid over a wire on a groove less drum, or for example, a wide belt over a wire on a groove less drum) drums and for the grooves, themselves.

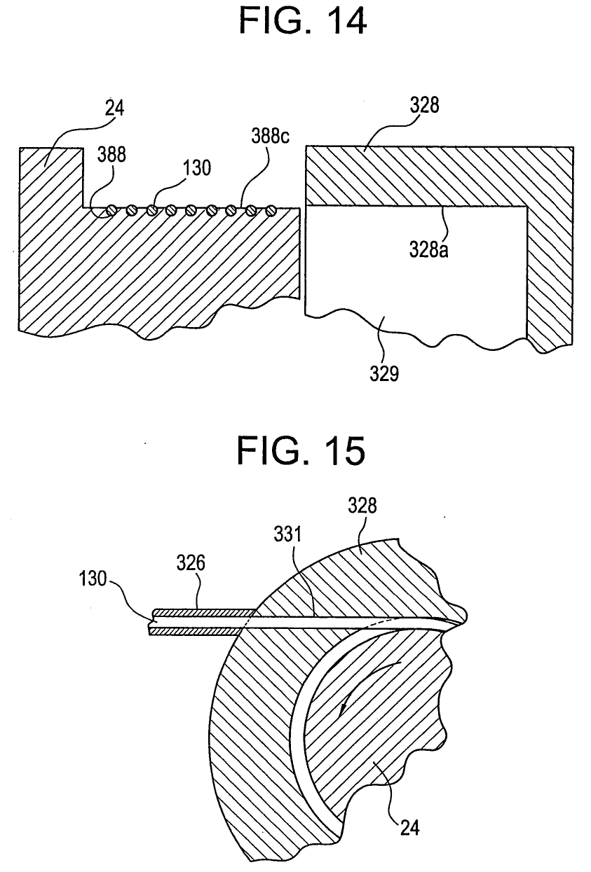

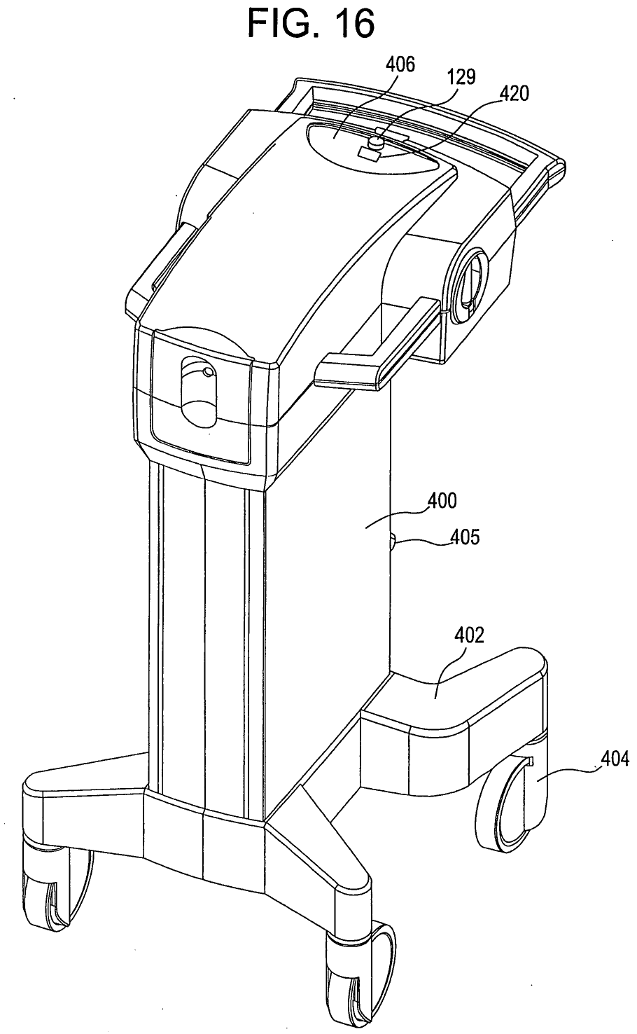

Referring to FIGS. 14-15, another exemplary embodiment for retaining a wire within a threaded groove 388 of a wire drum 24 is illustrated. The wire drum 24 is similar to that described above, except that the configuration is modified to accommodate a retaining cover 328 which is formed as a cylindrical member, having a cylindrical recess 329 formed therein. The retaining member 328 performs a similar function to that of the cable 128 above and acts as a cap, which is inserted over the wire drum 24 to retain the wire in the threaded groove 388. While above-described exemplary embodiment is illustrated, including a threaded groove, the retaining member 328 may simply hold a wire 130 on an outer circumferential surface of the wire drum 24, or it may hold a wire 130 in a groove (or grooves) or on a drum which is not threaded.

Referring now to FIG. 15, in one exemplary embodiment, the retaining member 328 is configured and dimensioned such that the inner side wall surface 328a forms a cylindrical sidewall having a diameter slightly larger than the diameter of the ridges 388c of the threaded groove 388. In this manner, upon insertion of the retainer 328 over the threaded groove, the wire 130 is slightly impinged against the threaded groove 388 and is thereby prevented from leaving the threaded groove 388.

A bore 331 may be formed through the retaining member 328 in tangential relationship with the surface of the drum 24. In this manner, the wire 130 is permitted to payout or exit the threaded groove 388 at a desired pre-determined angle upon rotation of the wire drum 24 as indicated by the arrow in FIG. 15.

Referring now to FIG. 16, an exemplary user interface will now be described. The present afterloader may be constructed as a simple, easy to use, highly portable, self-contained and highly versatile device. As shown, the exemplary afterloader is mounted at patient level on a rigid platform including a body 400 and base 402. The exemplary base includes multiple wheels 404 lockable with one or more foot pedals (not shown). The body may also include an emergency safe 405 such that a fractured wire or wire fragment may be conveniently disposed of and shielded.

In one embodiment, a user is assigned a key compatible with exemplary key selector switch 129. The key selector switch 129 may be spring loaded (such that it is disengaged prior to power up).

In another exemplary embodiment, a user interface portion 406 provides a plurality of visual indications. A first visual indicator 420 displays indication of the status of a power up diagnostic check. For example, the first visual indicator 420 may display a three digit numeric display allowing for code displays of 000 to 999. The first visual indicator 420 may also indicate switching into an accounting mode upon activation of each drum such that the first visual indicator will keep track of the number of times a particular wire has been used or the number of uses a particular wire has left. A second visual indicator is preferably set to provide advance notice of wire replacement (in accordance to the tracking of the first visual indicator) or to indicate that battery power is low and is switching to an auxiliary power source. When the number of uses of a particular wire is exhausted, the exemplary key selector switch 129 disengages such that it may only be accessed by a service key, which allows access to the interior of the afterloader and to cycle reset switches or other service concerns, such as power sources, among others.

In another exemplary embodiment, the key selector switch 129 has five positions. In a first position, the device is off. In a second position, a visual indication of the number of wire cycles (measured with the first completed crank cycle of each use) of drum 24 remaining is provided. In the exemplary embodiment, the key selector switch 129 moves through the second position to reach a third position, which activates drum 24 by removing the drum lock 117 discussed in relation to FIG. 4. In a fourth position, a visual indication of the number of wire cycles (measured with the first completed crank cycle of each use) of drum 50 remaining is provided. In another exemplary embodiment, the key selector switch 129 moves through the fourth position to reach a fifth position, which activates drum 50 by removing the drum lock 119 discussed in relation to FIG. 4. The above described key selector switch 129 and drum locks 117, 119 prevent both wires from even partially deploying at the same time.

A third visual indicator provides warning that a wire is in use or has not returned completely to a parked (home) position. The key selector switch 129 will preferably set to a neutral position (corresponding to positions two and four) or the off position, only when the extended wire is completely retracted and locked in place. In one exemplary embodiment, the key selector switch 129 is set to off or to neutral prior to allowing selection of another wire. For example, while the wire of drum 24 is played out (the key selector switch 129 is set to a third position), the key selector switch 129 will preferably not set to the second position (or any other position) until the drum is completely retracted. At that point, the key selector switch may automatically switch to the second position, the drum lock 117 is engaged, and the revised number of wire cycles may be displayed.

In another exemplary embodiment, while the key selector is set to the third or fifth position, that is, when either of the wire drums/cranks are activated, the first visual indicator displays the distance traveled by the tip of the wire.

Alternative embodiments not described are considered within the scope of the present disclosure. For example, the containment capsule 14 is described by FIG. 1 in a vertical orientation. A horizontal orientation is contemplated. Similarly, an embodiment wherein one or more containment capsules 14 or one or more paths 160 through the containment capsule 14 are provided to accommodate multiple sources. Also, FIG. 1 illustrates an embodiment wherein two cranks 22 are disposed on opposite sides of the afterloader. The present disclosure also contemplates an orientation and configuration where either the two cranks are positioned on the same side, or where a single crank selectively drives one or more drums via a mechanical or electrical selector switch.

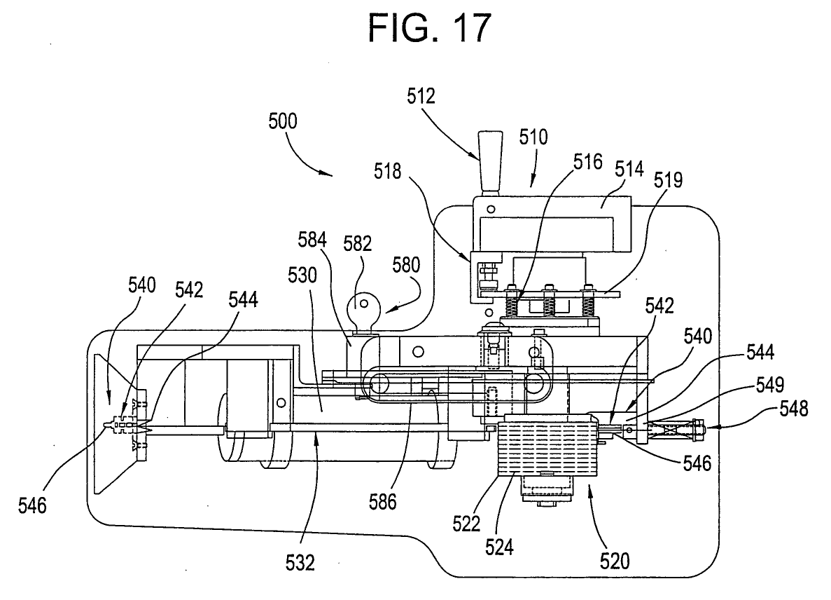

One such exemplary embodiment is illustrated with reference to FIG. 17. A manual afterloader is illustrated generally at 500. Afterloader 500 generally comprises a cranking mechanism, shown generally at 510, a first wire drum, shown generally at 520, selectively engageable with the cranking mechanism 510, a shield capsule 530, and a wire exit path, shown generally at 540. In the illustrated exemplary embodiment, the cranking mechanism includes a handle 512, a crank 514, a clutch mechanism 516 and a manual braking mechanism, shown generally at 518. Handle 512, crank 514, clutch 516 and braking mechanism 518 are similar to handle 46, crank 22, clutch 32 and braking mechanism 47 as illustrated in the exemplary embodiments of FIGS. 1 and 3. Braking mechanism 518 is illustrated in FIG. 17 in a disengaged position relative to braking disc 519, which is mounted to a cranking driveshaft (not shown).

In the exemplary embodiment illustrated in FIG. 17, the shield capsule 530 is relatively smaller than shield capsule 14 of FIG. 1, includes a linear pathway, shown generally at 532, and is constructed of a material having characteristics sufficient to block beta radiation.

Referring still to the exemplary embodiment illustrated in FIG. 17, a floating wheel 542 of a tensioning assembly, shown generally at 540, is illustrated. The floating wheel 542 is similar to the floating wheel 78 in the exemplary embodiment of FIG. 2, except that the floating wheel support member 544 is bounded on one side and open on the other side, thus facilitating replacement of cable 546, when necessary. Tensioning mechanism, shown generally at 548, is mounted to frame extension 549 and provides variable tension on floating wheel 542.

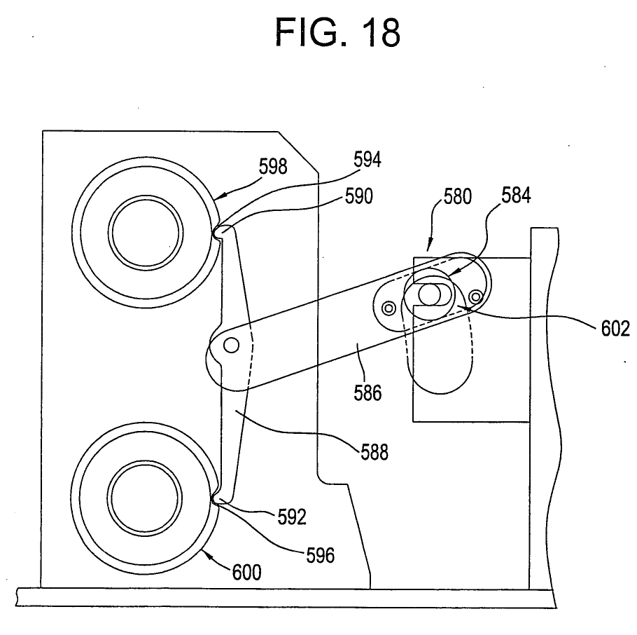

Referring still to exemplary embodiment illustrated by FIG. 17, a locking mechanism is provided generally at 580. The locking mechanism generally comprises a key 582, a locking cylinder 584, and a locking lever 586 engageable with the wire drum 520. The locking mechanism 580 may provide for locking of a single drum 520, which would otherwise be driven by cranking member 514, or it may provide for selective or concurrent locking of multiple drums (See FIG. 18), either selectively driven or concurrently driven by cranking mechanism 514. The locking mechanism 580 will be described in greater detail with reference to FIG. 18, below.

Referring still to the exemplary embodiment illustrated by FIG. 17, the wire drum 520 is illustrated as a grooved drum including at least one groove 522 and a wire 524 received therein. While a grooved drum 520 is illustrated as an exemplary embodiment, it should be noted that while the grooves 522 are an advantageous feature, a groove-less design may perform an equivalent useful purpose by itself or, for example, where a belt (not shown, but described with reference to FIGS. 12-13) or casing (not shown, but described with reference to FIGS. 14-15) are provided.

Referring still to the exemplary embodiment illustrated by FIG. 17, the wire exit path, shown generally at 540, may include an exit guide, shown generally at 542. In one exemplary embodiment, the wire exit path includes a wire funnel 544, which may be a symmetric or asymmetric guide for feeding a wire or wires into a proximal region 546 of the exit guide 540. The wire funnel 544 finds particular application where multiple drums (configured in a vertically stacked, horizontally stacked or random arrangement) are driven by the manual cranking mechanism and fed to the same exit path 540.

Referring now to FIG. 18, an exemplary locking mechanism 580 for a dual drum afterloader is illustrated, generally comprising a key 582 (shown in FIG. 17), a cylinder 584, a locking lever 586 and a swing arm lever 588. The swing arm 588 includes locking protrusions 590 and 592, which in locked positions, engage recesses 594 and 596 on wire drums 598 and 600 respectively. FIG. 18 shows the cylinder 584 at a first, locked position, shown generally at 602, which sets both locking protrusions 590, 592 within recesses 594, 596 respectively, and prevents both wire drums 598, 600 from rotating.

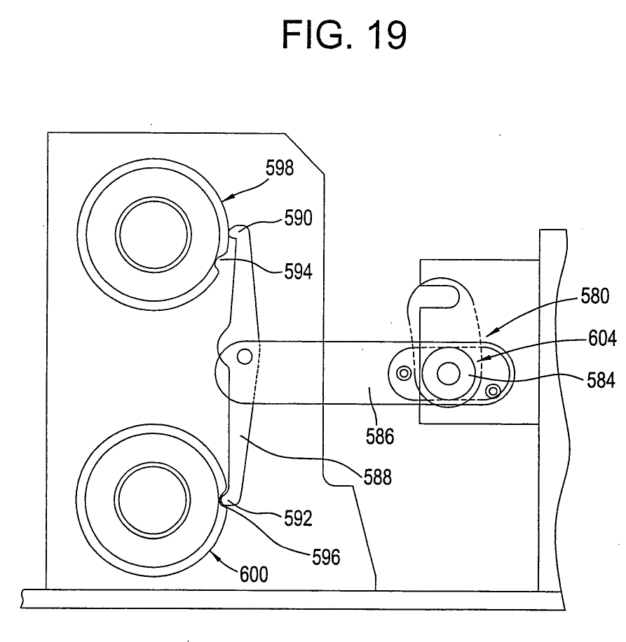

Referring now to FIG. 19, cylinder 584 is illustrated in a second, unlocked position 604, which in an exemplary embodiment, sets locking protrusion 592 within recess 596 of wire drum 600. In is generally advantageous to initially lock out a source wire drum and permit operation of a test wire drum such that patency of a source wire path may first be tested by a test wire. Subsequently, it may be advantageous, upon full retraction of the test wire, to lock out the test wire drum and permit treatment by a radioactive source wire. The exemplary embodiment in FIG. 19 illustrates such an initial, unlocked configuration, whereby a source wire drum 600 is initially locked and whereby a test wire drum 598 is free to rotate.

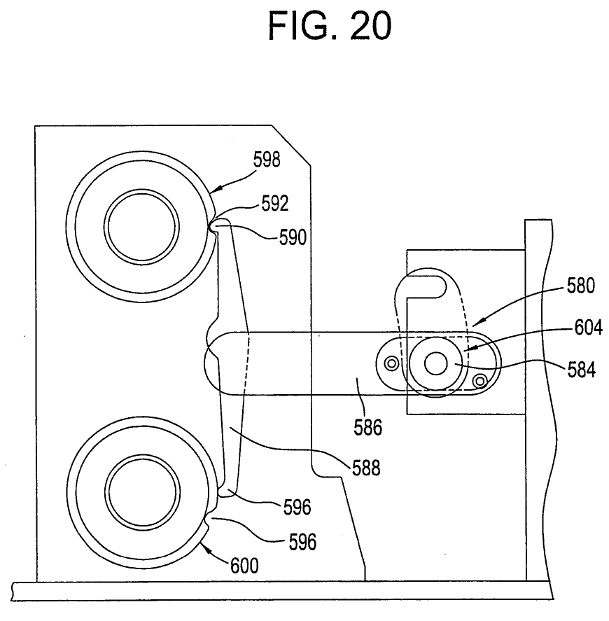

In another embodiment, the width of recesses 594, 596 are substantially configured to the negative geometry of the width of locking protrusions 590, 592, respectively. The wire drums 598, 600 may also be configured to displace laterally by thread engaged on a driveshaft, or by other equivalent mechanisms. Thus, the locking protrusions 590, 592 may be configured to only engage the recesses 594, 596 at single rotary position during travel of the wire drums 598, 600. In such an arrangement, or in an equivalent arrangement, the drum lockout positions can, for example, be set to provide that lockout will only occur on full retraction of the wire onto drums 598, 600. Further, the swing arm 588 can be biased to displace, upon full retraction of test wire onto drum 598, to swing into a position as described by FIG. 20.

Referring now to FIG. 20, the swing arm 588 is shown in a second position, where locking protrusion 590 is engaged with recess 592 on test wire drum 598. In contrast, locking protrusion 596 is illustrated as disengaged with recess 596 on source wire drum 600. In such a configuration, test wire drum 598 is locked in a retracted wire position (e.g., such that the test wire is removed from the exit path 540) and locking protrusion 596 is disengaged to allow operation of the source wire drum 600.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the invention. For example, the present disclosure similarly contemplates an embodiment wherein two cranks disposed on opposite sides of the afterloader each drive two drums, wherein the mechanical selector switch locks out the remaining drums allowing one of the four drums to be selectively driven. Additionally the temporary braking member need not be limited to a mechanical brake (including the pivoting crank handle), but may be electronically actuated. Other differences may be provided without affecting the overall functioning and wire retaining structure of the disclosed manually operated afterloader. All such changes and modifications are intended to be included within the scope of the present disclosure.

What is claimed is:

1. A manually operated afterloader for use with a radioactive elongated source wire, the source wire having a relatively short radioactive portion and a relatively long non-radioactive portion, comprising:
a base;
a shield capsule defining a pathway therethrough which receives a portion of a source wire,
a source wire drum rotatably mounted with respect to the base, the drum having a circumference and receiving a non-radioactive portion of a source wire;
a manually operable cranking mechanism mounted to the base and engagable with the drum to rotate the drum, the cranking mechanism, when operated driving a non-radioactive portion of the source wire off of the drum such that the radioactive portion of the source wire extends out of the shield capsule.

2. The afterloader as recited in claim 1, further comprising a cable mounted on the source wire drum such that the cable biases the non-radioactive portion of the source wire along the circumference of the drum.

3. The afterloader as recited in claim 2, wherein the cable is a flexible belt, and wherein the flexible belt is mounted around a first tension wheel biasing a portion of the flexible belt into engagement with the drum and a second tension wheel tensioning the flexible belt.

4. The afterloader as recited in claim 3, wherein the first tension wheel is fixed relative to the base plate and the second tension wheel is movable relative to the base plate so as to tension the flexible belt.

5. The afterloader as recited in claim 4, wherein the second tension wheel is mounted on a spring biased yoke mounted with respect to the base plate.

6. The afterloader as recited in claim 1, further comprising a guide tube positioned between the source wire drum and the shield capsule such that one end of the guide tube is aligned with a tangent of the source wire drum and an opposite end of the guide tube is aligned with one end of the pathway through the shield capsule.

7. The afterloader as recited in claim 1, wherein the pathway is non-linear.

8. The afterloader as recited in claim 1, wherein the cranking mechanism includes a drive shaft rotatably mounted with respect to the base plate, a crank affixed to one end of a drive shaft and a slip clutch operatively connected to the drive shaft.

9. The afterloader as recited in claim 8, wherein the cranking mechanism further includes a crank handle movable between a first position allowing rotation of the drive shaft and a second position frictionally restraining the drive shaft from being rotated.

10. The afterloader as recited in claim 1, further comprising a temporary stop engagable with the drum, the temporary stop halting rotation of the drum after one or more predetermined amounts of rotation of the drum.

11. The afterloader as recited in claim 1, wherein the drum further comprises a first stop block limiting the amount of rotation of the drum in a first direction and a second stop block limiting the amount of rotation of the drum in a second direction.

12. The afterloader as recited in claim 1, further comprising a drum lock engageable with a portion of the drum, the drum lock when in an engaged position, preventing movement of the drum.

13. The afterloader as recited in claim 12, wherein the circumference of the drum includes a threaded groove, the threaded groove receiving a non-radioactive portion of the source wire.

14. The afterloader as recited in claim 1, further comprising a second drum operatively associated with the cranking mechanism, wherein a selector permits rotation of one of the first and second drums.

15. The afterloader as recited in claim 14, further comprising first and second drum locks respectively engageable with the first and second drums, wherein the selector is engageable with the first and second drum locks to selectively prevent rotation of the drums.

16. A manually operated afterloader, comprising:
a base;
a shield capsule defining a pathway therethrough which receives a portion of a source wire;
a first drum rotatably mounted with respect to the base, the drum having a circumference and receiving a non-radioactive portion of a source wire;
a second drum rotatably mounted with respect to the base, the second drum having a circumference and receiving a portion of a second wire;
a first manually operable cranking mechanism mounted to the base and engagable with the first drum to rotate the first drum, the cranking mechanism, when operated driving a non-radioactive portion of the source wire off of the drum such that a radioactive portion of the source wire extends out of the shield capsule; and
a second manually operable cranking mechanism mounted to the base and engagable with the second drum to rotate the second drum, the cranking mechanism, when operated driving a portion of the second wire off of the drum such that an end portion of the second wire extends out of a channel within the afterloader.

17. The afterloader as recited in claim 16, further comprising first and second cables mounted on the first and second drums such that the cables bias respectfully the non-radioactive portion of the source wire and a portion of the second wire along the circumferences of the first and second drums.

18. The afterloader as recited in claim 17, wherein the cables are flexible belts, and wherein the flexible belts are mounted, on the first drum around a first tension wheel biasing a portion of the first flexible belt into engagement with the first drum and a second tension wheel tensioning the first flexible belt, and on the second drum, around a third tension wheel biasing a portion of the second flexible belt into engagement with the second drum and a fourth tension wheel tensioning the second flexible belt.

19. The afterloader as recited in claim 18, wherein the first and third tension wheels are fixed relative to the base plate and the second and fourth tension wheels are movable relative to the base plate so as to tension the first and second flexible belts, respectfully.

20. The afterloader as recited in claim 19, wherein the second and fourth tension wheels are mounted on first and second spring biased yokes, respectfully, mounted with respect to the base plate.

21. The afterloader as recited in claim 16, further comprising a guide tube positioned between the first drum and the shield capsule such that one end of the guide tube is aligned with a tangent of the first drum and an opposite end of the guide tube is aligned with one end of the pathway through the shield capsule.

22. The afterloader as recited in claim 16, wherein the pathway is non-linear.

23. The afterloader as recited in claim 1, wherein the each of the first and second cranking mechanisms include a drive shaft rotatably mounted with respect to the base plate, a crank affixed to one end of a drive shaft and a slip clutch operatively connected to the drive shaft.

24. The afterloader as recited in claim 23, wherein each of the cranking mechanisms further include a crank handle movable between a first position allowing rotation of the drive shaft and a second position frictionally restraining the drive shaft from being rotated.

25. The afterloader as recited in claim 16, wherein at least one of the first and second drums includes a temporary stop engagable with the drum, the temporary stop halting rotation of the drum after one or more predetermined amounts of rotation of the drum.

26. The afterloader as recited in claim 16, wherein the at least one drum further comprises a first stop block limiting the amount of rotation of the drum in a first direction and a second stop block limiting the amount of rotation of the drum in a second direction.

27. The afterloader as recited in claim 16, further comprising at least one drum lock engageable with a portion of the at least one of the first and second drums, the drum lock when in an engaged position, preventing movement of the drum.

28. The afterloader as recited in claim 27, wherein the circumference of at least one of the first and second drums includes a threaded groove, the threaded groove receiving a non-radioactive portion of a wire.

29. The afterloader as recited in claim 16, further comprising a third drum operatively associated with at least one of the first and second cranking mechanisms, wherein a selector permits rotation of one of the first, second or third drums.

30. The afterloader as recited in claim 29, further comprising a first, second and third drum lock engageable with the first, second and third drums, respectively, wherein the selector is engageable with the first, second and third drum locks to selectively prevent rotation of the drums.

31. The afterloader as recited in claim 16, further including one or more visual indicators, the visual indicators providing indication of which drum is active, the effective lifetime of a wire in cycles or an indication of the distance a wire has traveled.
